(19) United States
(12) Patent Application Publication
(10) Pub. No.: US 2023/0263981 A1
(43) Pub. Date: Aug. 24, 2023

(54) DEVICES, SYSTEMS, AND MASKS FOR CPAP, NON-INVASIVE VENTILATION, AND OXYGEN

(71) Applicant: THE REGENTS OF THE UNIVERSITY OF CALIFORNIA, Oakland, CA (US)

(72) Inventors: Jeremy Elliot Orr, San Diego, CA (US); Graham Jaffe Orr, Canyon Country, CA (US)

(21) Appl. No.: 18/018,191

(22) PCT Filed: Jul. 29, 2021
(86) PCT No.: PCT/US2021/043768

§ 371 Date: Jan. 26, 2023

Related U.S. Application Data
(60) Provisional application No. 63/058,356, filed on Jul. 29, 2020.

(51) Int. Cl. A61M 16/06; A61M 16/08; A61M 16/00

(52) U.S. Cl. CPC A61M 16/0616; A61M 16/0003; A61M 16/0816; A61M 2205/0216

(57) ABSTRACT

Device, systems, and methods relating to a mask interface are disclosed. For example, a mask interface may include a body and an interface coupled to the body. The body may include a nasal portion including an opening and a mouth portion including an open channel. The interface may be in contact with a subject’s face and may include a flexible material conformable to the subject’s face.

### CROSS REFERENCE TO RELATED APPLICATIONS

The present application is a U.S. national phase of PCT International Patent Application No. PCT/US2021/043768, filed Jul. 29, 2021 and titled “DEVICES, SYSTEMS, AND MASKS FOR CPAP, NON-INVASIVE VENTILATION, AND OXYGEN”, which claims priority to, and the benefit of, U.S. Provisional Pat. Application No. 63/058,356 filed on Jul. 29, 2020, and titled “DEVICES, SYSTEMS, AND MASKS FOR CPAP, NON-INVASIVE VENTILATION, AND OXYGEN”, which are incorporated herein by reference in their entireties.

### TECHNICAL FIELD

The present disclosure relates generally to a mask interface, and in particular, a mask interface that can be coupled to a positive pressure respiratory device.

### BRIEF SUMMARY OF THE DISCLOSURE

Disclosed are devices, systems, and methods that relate to a mask interface. A mask interface may include a body. The body may include a nasal portion. The nasal portion may include an opening. The nasal portion may be dimensioned to cover a nose region. The opening may be couplable with a positive pressure respiratory device. The body may include a mouth portion. The mouth portion may include an open channel. The mouth portion may be dimensioned to cover a mouth region. The open channel may be couplable with tubing. The body may include a rigid material. The mask interface may include an interface coupled to the body. The interface may be in contact with a subject’s face. The interface may include a flexible material conformable to the subject’s face.

In embodiments, the flexible material may be a porous medium or a viscoelastic solid with one or more holes.

In embodiments, the flexible material may include one or more of silicone and open cell foam.

In embodiments, the rigid material may include polycarbonate.

In embodiments, the interface may be coupled to the body with a bond joint or suction.

In embodiments, the body may include one or more internal passageways with a double-walled body. At least one of the one or more internal passageways may be coupled to the opening.

In embodiments, the interface may include a sealing feature isolating an interior of the body from an exterior environment of the mask interface and an internal passageway of the body.

In embodiments, the interface may include grooves on an exterior edge of the mask interface.

In embodiments, the body may include a facial adapter and a housing.

In embodiments, the tubing may be coupled to a suction generator. Applying suction via the suction generator may isolate an interior of the mask interface from an exterior of the mask interface.

Additional aspects of the present disclosure relate to a mask interface which includes a housing. The housing may include a first connector. The first connector may be couplable with a positive pressure respiratory device. The housing may also include an internal passageway coupled to the first connector. The housing may include a second connector. The second connector may be couplable with tubing from a suction generator. The housing may include a rigid material. The mask interface may include a facial adapter coupled to the housing and an interface. The mask interface may include an interface. The interface may be in contact with a subject’s face. The interface may include a flexible material conformable to the subject’s face.

In embodiments, the flexible material may be a porous medium or a viscoelastic solid with one or more holes.

In embodiments, the flexible material may include one or more of silicone and open cell foam.

In embodiments, the rigid material may include polycarbonate.

In embodiments, the interface may be coupled to the interface with a bond joint or suction.

In embodiments, the internal passageway may include a double-walled body.

In embodiments, the interface may include a sealing feature isolating an interior of the mask interface from an exterior environment of the mask interface and the internal passageway.

In embodiments, the interface may include grooves on an exterior edge of the mask interface.

In embodiments, applying suction via the suction generator may isolate an interior of the mask interface from an exterior of the mask interface.

Additional aspects of the present disclosure relate to a mask interface which includes a housing. The housing may include a first connector. The first connector may be couplable with a positive pressure respiratory device. The housing may include a second connector. The second connector may be couplable with tubing from a suction generator. The housing may include a rigid material. The mask interface may include a facial adapter coupled to the housing and an interface. The mask interface may also include the interface in contact with a subject’s face. The interface may include a flexible material conformable to the subject’s face to create a seal.

### BRIEF DESCRIPTION OF THE DRAWINGS

The technology disclosed herein, in accordance with one or more various embodiments, is described with reference to the following figures. The drawings are provided for purposes of illustration only and merely depict typical or example embodiments of the disclosed technology. These drawings are provided to facilitate the reader’s understanding of the disclosed technology and shall not be considered limiting of the breadth, scope, or applicability thereof. It should be noted that for clarity and ease of illustration these drawings are not necessarily made to scale.

FIG. 1 illustrates an example mask with tubing interconnect, in accordance with various embodiments of the present disclosure.

FIG. 2 illustrates an example mask interface, in accordance with various embodiments of the present disclosure.

FIG. 3 illustrates a component of the example mask, in accordance with various embodiments of the present disclosure.

FIG. 4 illustrates a component of the example mask, in accordance with one embodiment of the present disclosure.

FIG. 5 illustrates an example mask, in accordance with one embodiment of the present disclosure.

FIGS. 6A-D illustrates an example mask, in accordance with various embodiments of the present disclosure.

FIG. 7 illustrates an example computing component that may be used to implement features of various embodiments of the disclosure

The figures are not intended to be exhaustive or to limit the presently disclosed technology to the precise form disclosed. It should be understood that the presently disclosed technology can be practiced with modification and alteration, and that the disclosed technology be limited only by the claims and the equivalents thereof.

### DETAILED DESCRIPTION OF THE EMBODIMENTS

Masks are a component of several respiratory devices. For example, this may include positive airway pressure devices, continuous positive airway pressure (CPAP), bilevel positive airway pressure (BIPAP), supplemental oxygen devices, and/or other devices. Current masks for positive-pressure respiratory assistance (NIV, CPAP, BPAP) utilize pliable material at the interface, as well as straps, to keep the mask in place on the face and to try to maintain a good seal. Existing masks have leaks that are common during use. These leaks have been shown to decrease the effectiveness of various therapies. In the ICU, failure and/or leaks may lead to intubation. In addition, leaks may include exhaled droplets, which, under positive pressure, are propelled and/or aerosolized around the room which provide an infection risk. Similar issues apply to non-sealed oxygen masks as well.

In one example, due to a concern for the spread of infection (e.g., COVID-19 among other viruses and infectious diseases), BIPAP has been currently banned by many institutions. Instead of using such devices, patients who are decompensating are often intubated. This causes about a 50% mortality rate, which is in part related to infections, heavy sedation, and the like. While BIPAP has been shown to reduce mortality in more mild lung injury cases, sickest patients still need intubation. During the COVID-19 pandemic, there has been a reluctance to use BIPAP for non-COVID patients, which creates collateral damage. There is a similar prohibition on use of high flow oxygen due to non-sealed masks, despite evidence of the benefits of these devices.

The presently disclosed technology provides a novel suction mask interface utilizing suction. The mask may be shaped, dimensioned, or otherwise fit similar to a normal NIV/CPAP/BPAP device. This may allow the mask to interface with a positive or variable pressure respiratory device or other respiratory therapy such as supplemental oxygen. The mask may sit over the nose and/or mouth area, connecting to a standard hose/tubing that transmits positive airway pressure from an attached ventilator, pressure/flow generator device (i.e. sometimes called a “BIPAP” machine), and/or other respiratory therapies such as oxygen. In embodiments, the interface area between the mask and patient may include a channel to transmit negative suction pressure. In embodiments, this channel does not communicate with the positive pressure area. This suction channel may follow all along the mask-patient interface zone, with an area to attach to a hose/tubing that transmits pressure from an attached suction generator and/or controller (e.g. a hospital wall-suction system). In one example, suction may be applied via a channel at the interface between the skin and the mask. In embodiments, similar to what is described herein, the suction may be connected via tubing to a standard hospital wall suction controller. The presently disclosed technology provides several benefits, including the presently disclosed technology can maintain a mask seal onto the face; if the seal is disrupted, the presently disclosed technology helps re-establish the seal, if the seal is disrupted, the presently disclosed technology evacuates the leakage rather than allowing leaked air and/or other particulates to escape into the room.

In embodiments, the shape of the mask interface may be varied to fit a subject’s face. In some embodiments, different materials may be used for the interface, including silicone, and/or other soft materials. This may improve the seal and the suction level. In embodiments, the leak clearance may be optimized based on modifying channel dimensions, suction level, and/or other components of the mask interface. In some embodiments, the shape of the mask may be customized to each subject’s face, utilizing 3 dimensional facial scanning technologies or other sources of 3 dimensional face shape data, along with 3 dimensional printing of mask components and/or molds.

The presently disclosed technology may be used to deliver non-invasive ventilation (NIV), continuous positive airway pressure (CPAP), bilevel positive airway pressure (BPAP), non-invasive ventilation (NIV) or other similar forms of respiratory assistance such as supplemental oxygen. For such therapy, as discussed herein, maintenance of an airtight seal between the mask interface and the patient’s face is very important towards the effectiveness of therapy. The presently disclosed technology may utilize a suction, or partial vacuum, at the interface between a mask and a patient’s skin to maintain an airtight seal and prevent leaks. In addition, in the event of a gap developing between the mask and the patient, the suction provided by the presently disclosed technology may help to prevent any leakage from going into the room.

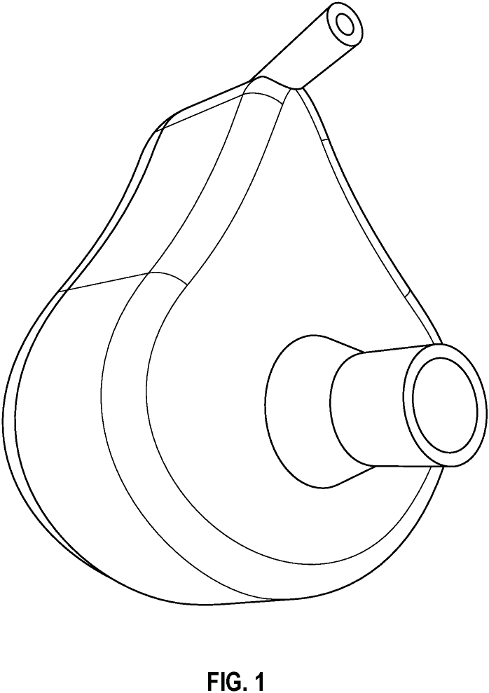

FIG. 1 illustrates an example mask interface with tubing interfaces, in accordance with various embodiments of the present disclosure. FIG. 1 may illustrate a perspective of the mask that is facing away from a subject during use. In embodiments, a first pathway may be used to supply positive pressure. For example, this first pathway may be the larger pathway. In some embodiments, a second pathway may be used to supply the suction. For example, this second pathway may be the smaller pathway. This second pathway may be referred to as a channel herein. The mask may be fit to interface between a positive-pressure respiratory device and a subject’s face. The mask may cover at least a subject’s nose and/or mouth.

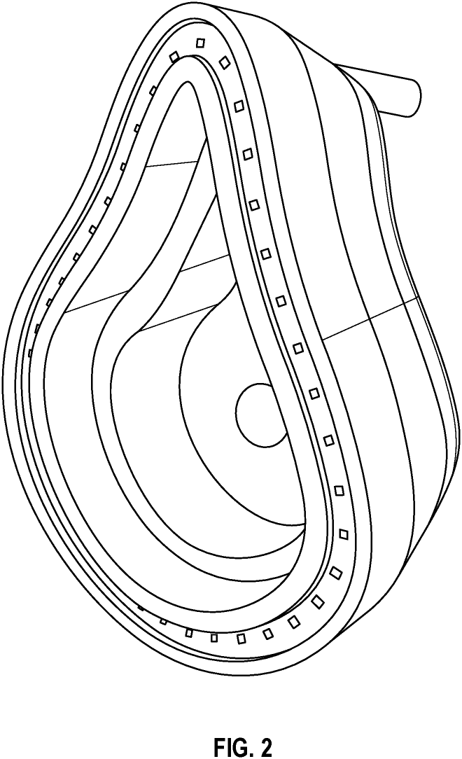

FIG. 2 illustrates an example mask interface, in accordance with various embodiments of the present disclosure. FIG. 2 may illustrate a perspective of the mask interface that is facing toward a subject during use. For example, this may be what interfaces directly with a subject’s face. In embodiments, the first material directly in contact with a subject may be a different material than the rest of the mask. For example, the first material may be softer and/or flexible to provide a suction against a subject’s face (e.g., silicone, etc.). The second material may be more rigid (e.g., polycarbonate, etc.).

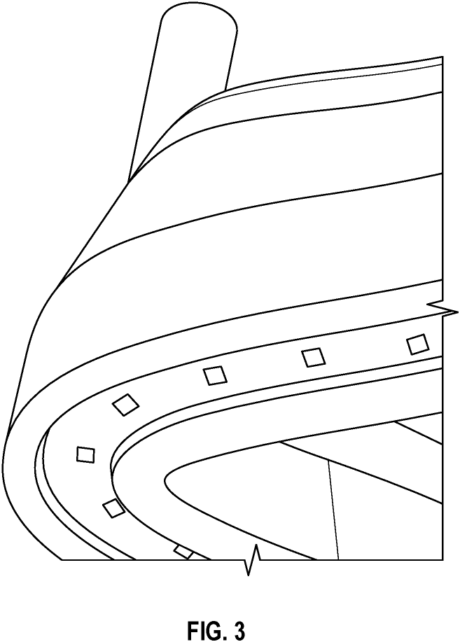

FIG. 3 illustrates a component of the example mask interface, in accordance with various embodiments of the present disclosure. This is an example of the mask interface near the nose and/or mouth region of the respiratory device where it contacts the skin, showing detail of the suction channel interface.

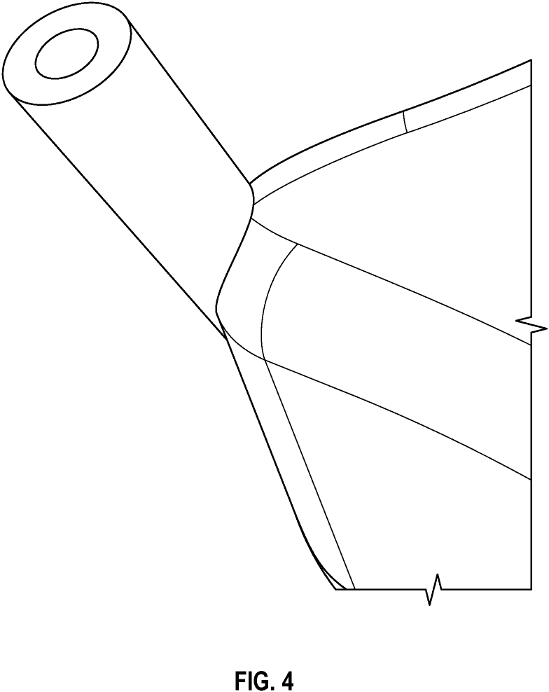

FIG. 4 illustrates a component of the example mask, in accordance with one embodiment of the present disclosure. The component may interconnect with tubing to provide air to a subject when the device is in use. In embodiments, this component may be used to apply suction during use.

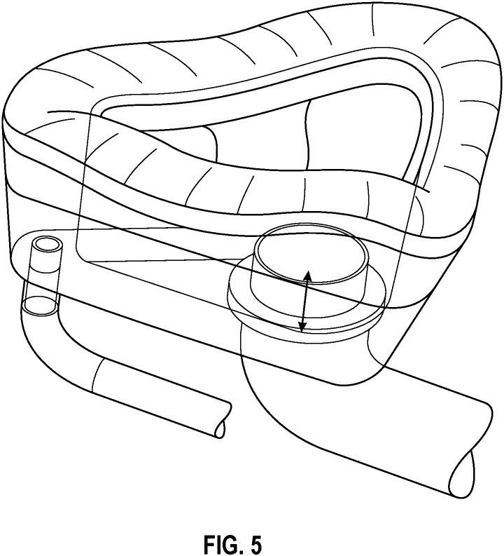

FIG. 5 illustrates an example mask, in accordance with one embodiment of the present disclosure. In one example of the mask, the portion of the mask interface in direct contact with a subject may be made of silicone. The silicone portion may be connected to the rest of the mask body using a bond joint, suction, and/or other mechanisms. The rest of the mask may be made of polycarbonate and/or other materials, including low-cost materials.

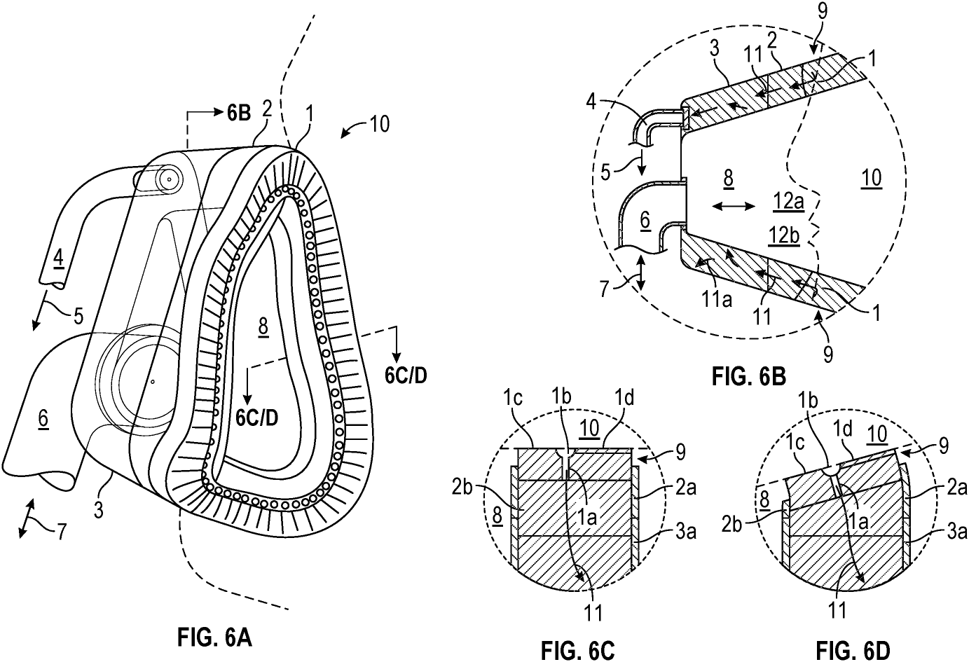

FIG. 6 illustrates an example mask device, in accordance with various embodiments of the present disclosure. Component 1 may provide a compliant interface, transmit suction, and seal against the subject’s face 10. The interface material may be viscoelastic solid with hole features or porous media such as open-cell foam. A partial vacuum may be transmitted through facial adapter 2 and housing 3 via internal passageways 11 within a double-walled body. Standard suction tubing 4 may connect to regulated vacuum supply 5. Within the airway region 8, respiration may be provided to/from nose 12a and mouth 12b via standard connector 6 to regulated respirator device 7, such as CPAP, BIPAP, NIV, supplemental oxygen, and/or other respiratory therapy. On the interface between device and face, sealing feature 1c may isolate airway region 8 from environment 9 and suction 11. Features 1a, 1b, and 1d may distribute the suction across the mask interface surface. Some embodiments may utilize grooves or texture within feature 1d to enhance suction and particle scrubbing effect near 9. Some embodiments permit components 1, 2, and 3 to be modular in nature, so that hospitals can digitally scan a subject’s face and then rapidly manufacture adapter 2 to join with other pre-fabricated components for a semi-universal solution.

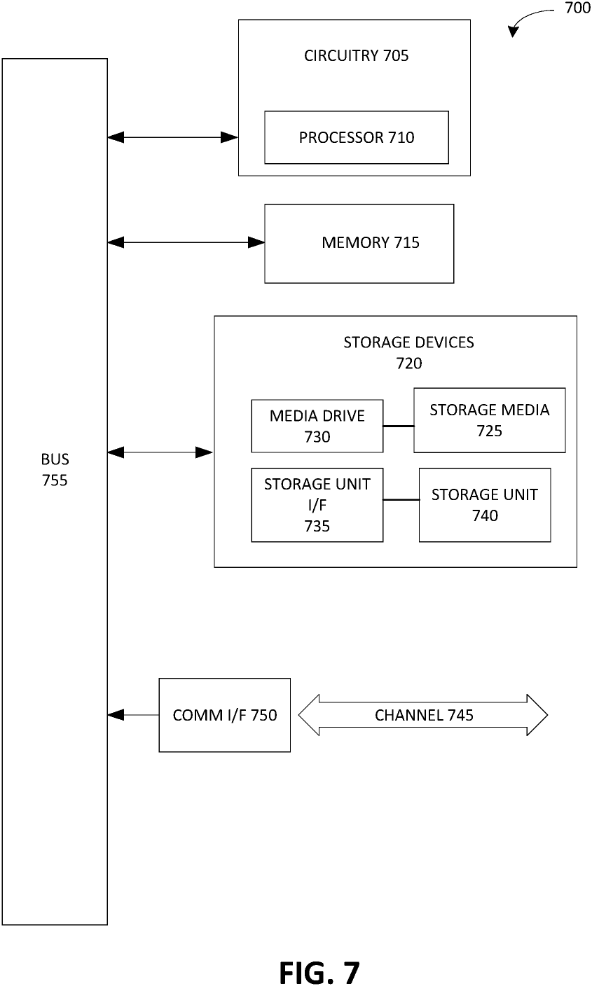

FIG. 7 illustrates example computing component 700, which may in some instances include a processor on a computer system (e.g., control circuit). Computing component 700 may be used to implement and/or monitor various features and/or functionalities of embodiments of the systems, devices, and methods disclosed herein. With regard to the above-described embodiments set forth herein in the context of systems, devices, and methods described with reference to FIGS. 1-6, including embodiments involving the control circuit, one of skill in the art will appreciate additional variations and details regarding the functionality of these embodiments that may be carried out by computing component 700. In this connection, it will also be appreciated by one of skill in the art upon studying the present disclosure that features and aspects of the various embodiments (e.g., systems) described herein may be implemented with respected to other embodiments (e.g., methods) described herein without departing from the spirit of the disclosure.

As used herein, the term component may describe a given unit of functionality that may be performed in accordance with one or more embodiments of the present application. In embodiments, a component may be implemented utilizing any form of hardware, software, or a combination thereof. For example, one or more processors, controllers, ASICs, PLAs, PALs, CPLDs, FPGAs, logical components, software routines, or other mechanisms may be implemented to make up a component. In implementation, the various components described herein may be implemented as discrete components or the functions and features described may be shared in part or in total among one or more components. In other words, as would be apparent to one of ordinary skill in the art after reading this description, the various features and functionality described herein may be implemented in any given application and may be implemented in one or more separate or shared components in various combinations and permutations. Even though various features or elements of functionality may be individually described or claimed as separate components, one of ordinary skill in the art will understand upon studying the present disclosure that these features and functionality may be shared among one or more common software and hardware elements, and such description shall not require or imply that separate hardware or software components are used to implement such features or functionality.

Where components or components of the application are implemented in whole or in part using software, in embodiments, these software elements may be implemented to operate with a computing or processing component capable of carrying out the functionality described with respect thereto. One such example computing component is shown in FIG. 7. Various embodiments are described in terms of example computing component 700. After reading this description, it will become apparent to a person skilled in the relevant art how to implement example configurations described herein using other computing components or architectures.

Referring now to FIG. 7, computing component 700 may represent, for example, computing or processing capabilities found within mainframes, supercomputers, workstations or servers; desktop, laptop, notebook, or tablet computers; hand-held computing devices (tablets, PDA’s, smartphones, cell phones, palmtops, etc.); or the like, depending on the application and/or environment for which computing component 700 is specifically purposed.

Computing component 700 may include, for example, one or more processors, controllers, control components, or other processing devices, such as a processor 710, and such as may be included in 705. Processor 710 may be implemented using a special-purpose processing engine such as, for example, a microprocessor, controller, or other control logic. In the illustrated example, processor 710 is connected to bus 755 by way of 705, although any communication medium may be used to facilitate interaction with other components of computing component 700 or to communicate externally.

Computing component 700 may also include one or more memory components, simply referred to herein as main memory 715. For example, random access memory (RAM) or other dynamic memory may be used for storing information and instructions to be executed by processor 710 or 705. Main memory 715 may also be used for storing temporary variables or other intermediate information during execution of instructions to be executed by processor 710 or 705. Computing component 700 may likewise include a read only memory (ROM) or other static storage device coupled to bus 755 for storing static information and instructions for processor 710 or 705.

Computing component 700 may also include one or more various forms of information storage devices 720, which may include, for example, media drive 730 and storage unit interface 735. Media drive 730 may include a drive or other mechanism to support fixed or removable storage media 725. For example, a hard disk drive, a floppy disk drive, a magnetic tape drive, an optical disk drive, a CD or DVD drive (R or RW), or other removable or fixed media drive may be provided. Accordingly, removable storage media 725 may include, for example, a hard disk, a floppy disk, magnetic tape, cartridge, optical disk, a CD or DVD, or other fixed or removable medium that is read by, written to or accessed by media drive 730. As these examples illustrate, removable storage media 725 may include a computer usable storage medium having stored therein computer software or data.

In alternative embodiments, information storage devices 720 may include other similar instrumentalities for allowing computer programs or other instructions or data to be loaded into computing component 700. Such instrumentalities may include, for example, fixed or removable storage unit 740 and storage unit interface 735. Examples of such removable storage units 740 and storage unit interfaces 735 may include a program cartridge and cartridge interface, a removable memory (for example, a flash memory or other removable memory component) and memory slot, a PCMCIA slot and card, and other fixed or removable storage units 740 and storage unit interfaces 735 that allow software and data to be transferred from removable storage unit 740 to computing component 700.

Computing component 700 may also include a communications interface 750. Communications interface 750 may be used to allow software and data to be transferred between computing component 700 and external devices. Examples of communications interface 750 include a modem or softmodem, a network interface (such as an Ethernet, network interface card, WiMediap, IEEE 77.XX, or other interface), a communications port (such as for example, a USB port, IR port, RS232 port Bluetooth® interface, or other port), or other communications interface. Software and data transferred via communications interface 750 may typically be carried on signals, which may be electronic, electromagnetic (which includes optical) or other signals capable of being exchanged by a given communications interface 750. These signals may be provided to/from communications interface 750 via channel 745. Channel 745 may carry signals and may be implemented using a wired or wireless communication medium. Some non-limiting examples of channel 745 include a phone line, a cellular or other radio link, an RF link, an optical link, a network interface, a local or wide area network, and other wired or wireless communications channels.

In this document, the terms “computer program medium” and “computer usable medium” are used to generally refer to transitory or non-transitory media such as, for example, main memory 715, storage unit interface 735, removable storage media 725, and channel 745. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions may enable the computing component 700 or a processor to perform features or functions of the present application as discussed herein.

While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent component names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the disclosed technology is described above in terms of various exemplary embodiments and implementations, it should be understood that the various features, aspects and functionality described in one or more of the individual embodiments are not limited in their applicability to the embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The term “respiratory device” is used generally to indicate any existing or future device that provides continuous or variable airway pressure, non-invasive ventilation (NIV), supplemental oxygen or other media such as nebulized and/or aerosolized medications and gas/liquid soluble therapies. These therapies may be provided by a myriad of physical machines, including hospital ventilators, home ventilators, home “BPAP” or “CPAP” devices, oxygen concentrators, high-flow oxygen and humidification devices, nebulizers, or other specialized devices for delivering inhaled medications.

What is claimed is:

1. A mask interface comprising:
a body comprising:
a nasal portion comprising an opening, wherein the nasal portion is dimensioned to cover a nose region, and wherein the opening is couplable with a positive pressure respiratory device; and
a mouth portion comprising an open channel, wherein the mouth portion is dimensioned to cover a mouth region, and wherein the open channel is couplable with tubing;
wherein the body comprises a rigid material; and
an interface coupled to the body, wherein the interface is in contact with a subject’s face, and wherein the interface comprises a flexible material conformable to the subject’s face.

2. The mask interface of claim 1, wherein the flexible material is a porous medium or a viscoelastic solid with one or more holes.

3. The mask interface of claim 1, wherein the flexible material comprises one or more of silicone and open cell foam.

4. The mask interface of claim 1, wherein the rigid material comprises polycarbonate.

5. The mask interface of claim 1, wherein the interface is coupled to the body with a bond joint or suction.

6. The mask interface of claim 1, wherein the body comprises one or more internal passageways with a double-walled body, and wherein at least one of the one or more internal passageways is coupled to the opening.

7. The mask interface of claim 1, wherein the interface comprises a sealing feature isolating an interior of the body from an exterior environment of the mask interface and an internal passageway of the body.

8. The mask interface of claim 1, wherein the interface comprises grooves on an exterior edge of the mask interface.

9. The mask interface of claim 1, wherein the body comprises a facial adapter and a housing.

10. The mask interface of claim 1, wherein the tubing is coupled to a suction generator, and wherein applying suction via the suction generator isolates an interior of the mask interface from an exterior of the mask interface.

11. A mask interface comprising:
a housing comprising:
a first connector, wherein the first connector is couplable with a positive pressure respiratory device;
an internal passageway coupled to the first connector; and
a second connector, wherein the second connector is couplable with tubing from a suction generator;
wherein the housing comprises a rigid material;
a facial adapter coupled to the housing and an interface; and
the interface, wherein the interface is in contact with a subject’s face, and wherein the interface comprises a flexible material conformable to the subject’s face.

12. The mask interface of claim 11, wherein the flexible material is a porous medium or a viscoelastic solid with one or more holes.

13. The mask interface of claim 11, wherein the flexible material comprises one or more of silicone and open cell foam.

14. The mask interface of claim 11, wherein the rigid material comprises polycarbonate.

15. The mask interface of claim 11, wherein the interface is coupled to the interface with a bond joint or suction.

16. The mask interface of claim 11, wherein the internal passageway comprises a double-walled body.

17. The mask interface of claim 11, wherein the interface comprises a sealing feature isolating an interior of the mask interface from an exterior environment of the mask interface and the internal passageway.

18. The mask interface of claim 11, wherein the interface comprises grooves on an exterior edge of the mask interface.

19. The mask interface of claim 11, wherein applying suction via the suction generator isolates an interior of the mask interface from an exterior of the mask interface.

20. A mask interface comprising:
a housing comprising:
a first connector, and wherein the first connector is couplable with a positive pressure respiratory device; and
a second connector, wherein the second connector is couplable with tubing from a suction generator;
wherein the housing comprises a rigid material;
a facial adapter coupled to the housing and an interface; and
the interface in contact with a subject’s face, wherein the interface comprises a flexible material conformable to the subject’s face to create a seal.
